(19) United States
(12) Patent Application Publication
(10) Pub. No.: US 2004/0052837 A1
(43) Pub. Date: Mar. 18, 2004

(54) Lipid conjugated anti-cancer drugs and methods of use thereof

(76) Inventors: William Stillwell, Indianapolis, IN (US); Mustapha Zerouga, Indianapolis, IN (US); Laura J. Jenski, Milton, WV (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/602,978

(22) Filed: Jun. 24, 2003

Related U.S. Application Data
(60) Provisional application No. 60/392,117, filed on Jun. 27, 2002.

(51) Int. Cl. A61K 9/127; A61K 31/525

(52) U.S. Cl. 424/450; 514/560; 514/251

(57) ABSTRACT

Lipid based anti-proliferative agents and methods of use thereof are disclosed.

This application claims the benefit of U.S. Provisional Application 60/392,117 filed Jun. 27, 2002, the entire disclosure of which is incorporated by reference herein.

Pursuant to 35 U.S.C. §202(c) it is acknowledged that the U.S. Government has certain rights in the invention described herein, which was made with funds from the National Institutes of Health, Grant Number GM57371.

FIELD OF THE INVENTION

This invention relates to the fields of drug design and chemotherapy. Specifically, novel compositions are provided comprising anti-proliferative drugs conjugated to phosphatidyl cholines which may be used to advantage in the treatment and/or diagnosis of a variety of pathological disorders.

### BACKGROUND OF THE INVENTION

Several publications and patent documents are referenced in this application by numerals in parentheses in order to more fully describe the state of the art to which this invention pertains. Full citations for these references are found at the end of the specification. The disclosure of each of these publications and documents is incorporated by reference herein.

Natural and artificial chemotherapeutic agents are available for the treatment of various cancers. Of particular interest is the very toxic but nevertheless major anti-neoplastic drug currently in use, methotrexate (MTX), and the natural, biologically safe ω-3 fatty acid, docosahexaenoic acid (DHA, 22:6Δ4,7,10,13,16,19) . Although the molecular modes of action for MTX and DHA are complex and poorly understood, they undoubtedly function by different mechanisms.

MTX was initially described in 1948 by Farber et al.(1) as an antineoplastic agent and remains the major antimetabolite used for chemotherapy of many different cancers (2). MTX has been reported to block proliferation of a wide variety of human neoplastic cells(3) and is toxic at very low levels (2×10⁻⁸ M) (1). MTX affects rapidly dividing cells during the S phase (4,5) through inhibition of dihydrofolate reductase and 5-aminoimidazole-4-carboxamide ribonucleotide transformylase, enzymes involved in nucleotide synthesis (4). The drug is therefore an antifolate, interfering with the cell's ability to synthesize purines and thymidylic acid, which are essential for DNA synthesis and cell division.

Since at physiological pH 99% of the MTX molecules exist as dianions, crossing the anionic membrane surface is energetically unfavorable. As a result, resistance to MTX uptake has been reported for a variety of mammalian tumor cells (6). To counter this problem, several lipophilic MTX analogs have been synthesized and tested. In general, these analogs have either a lipophilic ester attached to the γ carboxyl or have increased the length of the chain attached to the MTX carboxyl group. A few of these compounds were shown to strongly inhibit dihydrofolate reductase (7) and, at high concentration, exhibited a similar therapeutic effect as MTX (8,9)

Another approach to increase MTX uptake was to make MIX derivatives that have the drug covalently associated with phospholipids. In the 1980s Kinsky and Hashimoto synthesized a series of novel lipophilic MTXs and tested their effect on mouse cell proliferation (10-12). Their derivatives attached MTX to the primary amine on the head group of dimyris-toylphosphatidylethanolamine (DMPE). The Δ-MTX-DMPE derivative completely inhibited cell proliferation and dihydrofolate reductase activity (11). Later, Williams et al. (13-14) reported that their similar MTX-DMPE derivatives were effective at suppressing joint inflammation in antigen-induced arthritis in the rat. The problem with the MTXγ derivatized to the PE head group amine is that the linking bond is not readily hydrolyzed in either human plasma (13) or in human leukemic T cell lymphoblasts (12).

Anti-cancer properties have also been attributed to the ω-3 class of fatty acids. Of particular interest is DHA, the longest (22 carbons), most unsaturated (six double bonds) and abundant ω-3 fatty acid commonly found in animal cells, (15) DHA has been associated with benefit for diverse human afflictions, (16) including cancer (17,18). Early epidemiologic evidence has strongly linked dietary ω-3 fatty acids with low incidence of several types of cancer and DHA has been successfully applied as an anticancer agent when added to the diet of many animals including humans, (19) incubated with cultured tumor cells as a free fatty acid (20) or fused to cells as DHA-containing phosphatidylcholine liposomes (21).

Due largely to their benign nature and tremendous versatility, liposomes are currently being employed as delivery systems for many drugs. One important feature of liposome delivery is the decreased amount of drug required per administration; less drug means a lower risk of undesirable side effects. Both MTX and DHA have been successfully incorporated into liposomes and subsequently administered to cells. In one report, Comisky and Heath (22) employed liposomes made from a variety of lipids to deliver MTX to cells in culture. They reported enhanced efficiencies over free drug of up to 10⁴-fold, but encountered problems with MTX leakage from the liposomes, particularly in the presence of serum. Similar leakage problems for MTX have been reported by others and have been partially overcome by incorporation of large levels of liposomal cholesterol (22-24). Although not very leaky to MTX, cholesterol-rich liposomes are also not efficiently endocytosed into cells.

### SUMMARY OF THE INVENTION

In accordance with the present invention, compositions and methods are provided for the treatment of cancer. In one embodiment, an anti-proliferative composition comprising a lipid conjugated to an ω-3 fatty acid and a chemotherapeutic agent suspended in a biologically compatible buffer is provided. The anti-cancer composition is further incorporated into liposomes. Preferably, the ω-3 fatty acid is selected from the group consisting of docosahexanoic acid (DHA) and eicosapentaenoic acid (EPA). The chemotherapeutic agent may be any agent that contains the appropriate functional group for coupling to the lipid of the compositon. In a preferred embodiment, the chemotherapeutic agent is selected from the group consisting of methotrexate, chlorambucil, and melphalan. The lipid moiety of the composition is preferably phosphatidylcholine or phosphatidylethanolamine. The anti-cancer agent and the omega-3 fatty acid may be incorporated interchangeably into the sn-1 or sn-2 positions of the lipid.

In a further embodiment of the invention, the liposome comprising the anti-cancer composition of the invention is studded with a targeting agent such as an antibody to facilitate tumor cell specific targeting.

Finally, methods of administering effective amounts of the foregoing anti-cancer compositions of the invention to a patient in need there of are disclosed.

### BRIEF DESCRIPTION OF THE DRAWINGS

FIGS. 1A and 1B show the structures of (FIG. 1A) MTX and (FIG. 1B)22:6, MTXγ-PC.

FIG. 2 is a chromatogram showing separation of reaction products. 22:6, MTXα-PC and 22:6, MTXγ-PC were synthesized as described hereinbelow. The reaction products were separated on thin-layer chromatography plates with the solvent system, chloroform: methanol: water: 28% aqueous NaOH (65:20:3:0.15, v/v/v/v). The products were visualized by iodine vapors. Lane 1 contains the total reaction mixture. Lane 2 is a control containing the entire reaction mixture without 22:6, OH PC.

FIG. 3 shows the UV spectra of MTX-PC products. Absorbance spectra of (A) MTX (8.8 μM in 10 mM NaHP0₄, pH 7.4), (B) 18:0. MTXγ-PC (13 μM) and (C) 22:6, MTXγ-PC (9.8 μM). The MTXγPCs are insoluble in water and so were incorporated into small unilamellar vesicles made from 18:0,18:1 PC in 10 mM NaHP0₄, pH 7.4.

FIG. 4 shows the results of fatty acid chromatographic analysis of (A) 22:6, MTXγ-PC, (B) 10:0, MTXγ-PC and (C) 10:0, 22:6 PC.

FIG. 5 is a chromatograph showing 22:6, MTXγ-PC hydrolysis by phosphollpase _{A2}. Lipid vesicles were made from 18:0,18:1 PC and 22:6, MTXγ-PC (9/1, mol:mol, 1 mM) as described below. The reaction was conducted at 27° C. and initiated at zero time by adding phospholipase A₂ (2 μg/ml) to the vesicles. The reaction was terminated at the times shown by the addition of solvent; the organic and aqueous phases were isolated, and the reaction products separated by thin-layer chromatography as described in FIG. 2. Lane 1: control, time zero, organic phase; Lane 2: reaction time 20 min, organic phase; Lane 3: reaction time 60 min, organic phase; and Lane 4: reaction time 60 min, aqueous phase.

FIGS. 6A and 6B are a pair of graphs showing inhibition of cell proliferation by MTX and by DHA. T27A cells were cultured for 24 h in the presence of MTX (FIG. 6A) or DHA (FIG. 6B), and proliferation was assessed by the incorporation of [³H]-thymidine. Mean±SE, n=6.

FIG. 7 is a graph showing inhibition of cell proliferation by liposomes containing 18:0, 22:6 PC. T27A cells were cultured for 24 h with 1 mM lipids sonicated into small unilamellar vesicles (liposomes). The bulk of the lipid bilayer was 18:0, 18:1 PC/18:0,18:1 PE (9:1, mol/mol) into which various concentrations (up to an additional 15 mol %, i.e., 150 μM) of 18:0,22:6 PC were added. Proliferation was estimated by [³H]-thymidine incorporation (mean±SE, n=6).

FIG. 8 is a graph showing that 22:6, MTXγ-PC is a more potent inhibitor of cell proliferation than 18:0, MTXγ-PC. T27A cells were cultured for 24 h with liposomes made from 1 mM lipids [18:0,18:1 PC/18:0,18:1 PE (9:1, mol/mol)) containing up to an additional 0.43 mol % (4.2 μM) of an MTXγ-PC. Proliferation was estimated by [³H]thymidine incorporation (mean±SE, n=6).

### DETAILED DESCRIPTION OF THE INVENTION

Phospholipids comprise the basic structural element of biological membranes. These lipids are composed of a polar head group (phosphorylcholine being the major one found in mammals) and two acyl chains that are attached o the head group at positions designated sn-1 and sn-2. The polar head group interacts with the surrounding water while the acyl chains are sequestered into the membrane interior that is devoid of water.

In accordance with the present invention, novel lipid based conjugates for use in the treatment of cancer are provided. Lipids, including but not limited to phophatidylcholine and phosphatidylethanolamine are conjugated at the sn-1 or sn-2 position to omega-3 fatty acids such as docosahexanoic acid and eicosapentaenoic acid. The composition further comprises an anti-cancer drug having the appropriate functional group conjugated to the lipid at the sn position not occupied by the omega-3 fatty acid. The conjugates of the invention are then incorporated into liposomes for administration to the patient. There are a variety of combinations of the above described reagents that may be synthesized in accordance with the methodology provided in the following example sections.

In one embodiment of the invention, the synthesis and characterization of a lipophilic phosphatidylcholine containing the ω-3 fatty acid docosahexaenoic acid (DHA) and the cytotoxic drug methotrexate (MTX) is provided. This novel phospholipid combines the fatty acid's and the drug's anticancer activities in a molecule amenable to a liposome bilayer for safe, simultaneous delivery of the two agents. Two phosphatidylcholines were synthesized from 1-stearoyl or 1-docosahexaenoyl, 2-hydroxy-sn-glycero-3-phosphocholine, to contain MTX in the sn-2 position and either stearic acid or DHA in the sn-1 position. The products contain fatty acid, MTX and phosphorus (1:1:1) and the MTX was released by phospholipase A₂ consistent with the proposed phospholipid structure. The predominant product linked MTX to the glycerol moiety through MTX's γ-carboxyl group. Liposomes composed of 1-stearoyl, 2-oleoyl phosphatidylcholine plus 1-stearoyl, 2-oleoyl phosphatidylethanolamine and various con-contrations of the novel phospholipids caused dose-dependent inhibition of murine leukemia cell proliferation in culture. The DHA and MTX-containing phosphatidylcholine was more effective than that containing stearic acid, and DHA appeared to synergize with MTX when they were added as free agents or covalently linked in the phospholipid. These data show the feasibility of synthesizing, and the inhibitory activity of phosphatidylcholine with DHA in the sn-1 position and MTX in the sn-2 position, and suggest the compound's potential use in cancer chemotherapy.

In a particularly preferred embodiment of the invention, the composition further comprises a suitable physiologically compatible buffer. Buffers suitable for this purpose are known in the art and include, but are not limited to, phosphate buffered saline (PBS), 3-[N-Morpholino]propanesulfonic acid (MOPS) and 3-[N-Morpholino]ethanesulfonic acid (MES). The compositions of the invention may be administered both systemically as well as injected directly at a disease site.

While methotrexate is exemplified herein, the present invention encompasses additional therapeutic agents which may be conjugated to the lipid moiety via the appropriate functional groups. Suitable therapeutic agents are those capable of acting on a cell, organ or organism to create a change in the functioning of the cell, organ or organism, including but not limited to pharmaceutical agents or drugs. Such agents include a wide variety of substances that are used in therapy, immunization or otherwise are applied to combat human and animal disease. Such agents include but are not limited to analgesic agents, anti-inflamatory agents, antibacterial agents, antiviral agents, antifungal agents, antiparasitic agents, tumoricidal or anti-cancer agents, proteins, toxins, enzymes, hormones, neurotransmitters, glycoproteins, immunoglobulins, immunomodulators, dyes, radiolabels, radio-opaque compounds, fluorescent compounds, polysaccharides, cell receptor binding molecules, anti-inflammatories, anti-glaucomic agents, mydriatic compounds and local anesthetics.

As mentioned previously, anti-neoplastic agents are preferred for use as biological agents in the compositions of the invention. Representative examples include, but are not limited to paclitaxel, daunorubicin, doxorubicin, carminomycin, 4-epiadriamycin, 4-demethoxy-daunomycin, 11-deoxydaunorubicin, 13-deoxydaunorubicin, adriamycin-14-benzoate, adriamycin-14-actanoate, adriamycin-14-naphthaleneacetate, vinblastine, vincristine, mitomycin C, N-methyl mitomycin C, bleomycin A₂, dideazatetrahydrofolic acid, aminopterin, methotrexate, cholchicine and cisplatin. Representative antibacterial agents are the aminoglycosides including gentamicin. Representative antiviral compounds are rifampicin, 3′-azido-3′-deoxythymidine (AZT), and acylovir. Representative antifungal agents are the azoles, including fluconazole, macrolides such as amphotericin B, and candicidin. Representative anti-parastic compounds are the antimonials. Suitable biological agents also include, without limitation vinca alkaloids, such as vincristine and vinblastine, mitomycin-type antibiotics, such as mitomycin C and N-methyl mitomycin, bleomycin-type antibiotics such as bleomycin A₂, antifolates such as methotrexate, aminopterin, and dideaza-tetrahydrofolic acid, taxanes, anthracycline antibiotics and others.

The composition may further include a targeting group including but not limited to antibody, fragment of an antibody, protein ligand, polysaccharide, polynucleotide, polypeptide, low molecular mass organic molecule and the like. Such targeting groups can be linked covalently to the liposome, or can be non-covalently incorporated in the compositions, e.g., through hydrophobic, electrostatic interactions or hydrogen bonds. For example, many cancer cells overexpress the epidermal growth factor receptor. Accordingly, the liposomal compositions of the invention may be studded with an anti-epidermal growth factor receptor antibody to facilitate tumor specific targeting. The liposomes of the invention may be studded with a Herceptin® antibody to facilitate targeting to breast cancer tissue. Methods for synthesizing antibody-studded liposomes are described in U.S. Pat. No. 6,316,024, the entire disclosure of which is incorporated by reference herein.

The compositions of the present invention allow diverse routes of administration, including but not limited to parenteral (such as intramuscular, subcutaneous, intraperitoneal, and intraveneous), oral, otic, topical, vaginal, pulmonary, and ocular. These compositions can take the form of aqueous solutions, suspensions, micelles, vesicles, emulsions and microemulsions.

Conventional pharmaceutical formulations may be employed. When aqueous suspensions are required for oral use, the composition can be combined with emulsifying and suspending agents. For parenteral administration, sterile solutions of the composition are usually prepared, and the pH of the solutions are suitably adjusted and buffered. For intravenous use, the total concentration of solutes should be controlled to render the preparation isotonic. For ocular administration, ointments or droppable liquids may be delivered by well-known ocular delivery systems such as applicators or eyedroppers. Such compositions can include mucomimetics such as hyaluronic acid, chondroitin sulfate, hydroxypropyl methylcellulose or polyvinyl alcohol, preservatives such as sorbic acid, EDTA or benzylchronium chloride, and the usual quantities of diluents and/or carriers. For pulmonary administration, diluents and/or carriers will be selected to be appropriate to allow for formation of an aerosol.

The invention will be described hereinbelow with particular emphasis on the formulation of a chemotherapeutic composition, in accordance with the teachings herein, for the treatment of cancer. It should be understood, however, that the present invention has a substantially broader range of applications as indicated above.

The following examples are provided to illustrate different embodiments of the invention. They are not intended to limit the invention in any way.

### EXAMPLE I

The present example sets forth the synthesis and characterization of a novel lipid conjugate which is an effective anti-cancer agent. The following materials and methods are provided to facilitate the practice of the present invention.

Synthesis of the MTX-PCs

The MTX-PCs were synthesized by coupling either 1-stearoyl, 2-hydroxy-sn-glycero-3-phosphocholine (18:0, OH (lyso) PC) or 1-docosahexaenol, 2-hydroxy-sn-glycero-3-phosphocholine (22:6, OH (lyso) PC) with MTX (A6770; Sigma, St Louis, Mo.) in dry chloroform. The 18:0, OH PC was purchased from Avanti Polar Lipids (Alabaster, Ala.). To synthesize 22:6, OH PC, we hydrolyzed 1,2-didocosahexaenoyl-sn-glycero-3-phosphocholine (22:6, 22:6 PC), the synthesis of which is described below, with phospholipase A₂.

Synthesis and Purification of 22:6, 22:6 PC 22:6

22:6 PC was synthesized as we described previously (28) with some modifications. Briefly, 2 eq (300 μmol) of DHA (NuChek Prep, Elysian, Minn.) were dissolved in 5 ml of dry, doubly distilled N, N-dimethylformamide, and then mixed with L-α-glycero-phosphorylcholine (0.25 eq, 37.5 μmol), dicyclocarbodiimide (2.5 eq, 375 μmol), 4-dimethylaminopyridine (1 eq, 150 μmol) and the antioxidant butylated hydroxytoluene (BHT) (7 μmol). The resulting mixture was stirred for 17 h at room temperature, in the dark under nitrogen, after which the suspension was filtered, washed with chloroform, concentrated and resuspended in chloroform:methanol:water (5:4:1, v/v). The solution was then passed over a Rexyn 1300 column (29). The column eluant was separated as described by Folch et al. (30) and the resultant lower phase collected, dried and resuspended in chloroform. The chloroform solution was loaded onto a carboxymethyl cellulose column and eluted with stepwise increases of methanol in chloroform. The purity of the synthesized product was tested by silica gel thinlayer chromatography using chloroform:methanol: water (65:25:4, v/v/v) and by gas chromatography on a Shimadzu GC-17A chromatograph as previously described (28). The phospholipid content was determined using a spectrophotometric assay for total phosphorus after mineralization (31). The yield of 22:6, 22:6 PC was 80-85% relative to L-1α-glycero-phosphorylcholine; the product was stored at −80° C.

Synthesis and Purification 22.6, OH PC 22:6.

OH PC was produced by phospholipase _{A2} hydrolysis of multilamellar vesicles composed of 22:6, 22:6 PC. 22:6, 22:6 PC (1 mM) was hydrated in 2.5 mM. CaCl₂, 0.1 M Tris buffer, pH 8.0, to form multilamellar vesicles. The vesicles were sonicated for 10 min at 25° C. in a bathtype sonicator, after which the tubes were transferred into a shaking water bath and the reaction initiated by adding phospholipase _{A2} from Naja mossambica (2 μg/ml, P 7778; Sigma). The mixture was incubated for 20 min at 25° C. and terminated with the addition of chloroform:methanol (2:1, v/v). The lipids were extracted by the method of Folch et al. (30) and the organic phase was purified on carboxymethyl cellulose as described above. Purity of 22:6, OH PC was tested by thin-layer chromatography. Under these conditions, 70% of the 22:6, 22:6 PC was hydrolyzed. The hydrolyzed product was stored at −80° C.

Synthesis and Purification of 18.0, MTX-PC and 22.6, MTX-PC

18:0, OH PC (2 eq, 50 μmol) and MTX (1 eq, 25 μmol) were dissolved separately in 2 ml of dry, doubly distilled chloroform, mixed together for a final volume of 4 ml and stirred for 60 min at room temperature under nitrogen. Dicyclocarbodiimide (4 eq, 100 μmol) and 4-dimethylaminopyridine (2 eq, 50 μmol) were added sequentially to the solution. The resulting mixture was stirred for 24 h in the dark under nitrogen at room temperature. The synthesis of 22:6, MTX-PC from 22:6, OH PC was identical to that of 18:0, MTX-PC except that the antioxidant BHT (2.5. μmol) was added to the mixture and the time of incubation was 18 h.

Purification of the MTX-PCs

The product of each reaction was dried under nitrogen and suspended in water:acetone (1:1, v/v). After mixing, the sample was extracted with 2 volumes of ethyl acetate. The aqueous phase, which contained mainly MTX and lyso-PC, was re-extracted and the two organic phases were combined and concentrated. The MTX-PCs were purified on analytical thin-layer plates (Silica Gel, 60A, 0.2 mm thickness, with or without fluorescent indicator). The plates were developed in chloroform:methanol:water:28% aqueous NaOH (65:20:3:0.15, v/v/v/v). Reaction products were visualized by a combination of iodine, acid molybdate reagent and UV light. Two yellow MTX-containing bands that also gave a positive molybdate test for phosphorus were visible and both bands absorbed UV light. Bands from the plates that did not contain fluorescent indicator were individually scraped, suspended in chloroform:methanol (20:80, v/v), passed through a glass filter under vacuum, and stored at −80° C. Sample purity was assessed by two-dimensional thin-layer chromatography [solvent a: chloroform:methanol:acetic acid:28% aqueous NaOH (65:20:3:0.15, v/v/v/v); solvent b: chloroform: methanol:acetone:acetic acid:water (50:10:20:10:5, v/v/v/v/v)]. The phosphate/MTX ratio was calculated from phosphorus after total mineralization (31) and from MTX UV absorbance compared to a MTX standard curve.

Hydrolysis of MTX-PCs by pbospholipase A₂

Lipid vesicles were made from 18:0, 18:1 PC and either 18:0, MTXγ-PC or 22:6, MTXγ-PC (9/1, mol:mol, 1 mM) in 2.5 mM CaCl₂, 0.1 M Tris, pH 8.0. The hydrated lipid mixtures were first sonicated in a water bath-type sonicator for 10 min at 27° C., transferred to a 27° C. shaking water bath and hydrolysis was initiated at zero time by adding N. mossambica phospholipase A₂ (2 μg/ml) to the vesicles. The reaction was terminated at different times by adding chloroform:methanol (2:1, v/v). The water and chloroform phases were isolated and the reaction products were separated by thin-layer chromatography with the solvent system, chloroform: methanol:water:28% aqueous NaOH (65:20:3:0.15, v/v/v/v). The products were visualized by iodine vapors.

Fatty Acid Analysis

The MTXγ-PC products were hydrolyzed and methylated using sodium methoxide as described by Eder et al. (32). The fatty acid methyl esters were extracted with hexane:water (2:1, v/v) and the organic phase used for gas chromatographic analysis on a Shimadzu GC-17A gas chromatograph using a 0.25 mm ×30 m Stabilwax Capillary Column (Restek, Bellefonte, Pa.), an automatic sample injector and a flame ionization detector. The programmed temperature ramp was 3°/min from 180-240° C. and 1°/min from 240 to 245° C.

Biological Testing

Cell Line

The murine non-T non-B leukemia cell line T27A (ATCC, Rockville, Md., USA) was cultured in RPMI 1640 medium (Gibco/BRI, Gaithersburg, Md.) supplemented with 25 mM HEPES buffer, 2 mM glutamine, 100 U penicillin/ml, 100 μg streptomycin/ ml and 2 or 10% bovine calf serum (Hyclone, Logan, Utah) in a humidified 5% CO₂ atmosphere. Cell viability was determined by Trypan blue exclusion.

Preparation of Fatty Acid- and MTX-Supplemented Media

DHA (NuChek Prep, Elysan, Minn.) was introduced into RPMI 1640 medium supplemented with 1% (w/v) fatty acid-free bovine serum albumin (Sigma) as described by Spector and Hoak (33) with modifications we have reported (34). MTX was dissolved in RPMI medium, and both media were sterile-filtered and stored at −20° C.

Liposome Preparation

Liposomes were prepared fresh for each use. The bulk of the lipids was 18:0, 18:1 PC (up to 90 mol %), with 10 mol % 18:0, 18:1 PE added to augment fusion between liposomes and the cell-surface membrane. The control contained no additional lipids; the experimental groups contained up to an additional 15 mol % 18:0, 22:6 PC or up to 0.43 mol % 18:0, MTX-PC or 22:6, MTX-PC. The solvent was evaporated under nitrogen and the lipids dried under vacuum for 2 h. The lipids were rehydrated in RPMI medium, and multilamellar vesicles were prepared by vortexing 1 ml of lipids at a concentration of 2 mg/ml. Liposomes (small unilamellar vesicles) were created by sonicating the multilamellar vesicles for 15 min on ice under nitrogen with a Heat Systems W380 disruptor (Farmingdale, N.Y.) set at 1 s cycle time, 60% duty and 3-7% output. Liposome suspensions were centrifuged at low speed (3700 r.p.m.; 2300 g) to remove particulate material and sterile filtered through a 0.2-μm filter. An aliquot of each liposome preparation was tested by thin-layer chromatography to assure that hydrolysis had not occurred.

Treatment of Cells in Culture

T27A cells, 10⁴ cells/ well, were introduced into 96-well flat-bottom tissue culture plates in medium containing 2% bovine calf serum, and the agents (DHA, MTX and liposomes) were added at various concentrations into six, replicate wells at each concentration. All concentrations given are final concentrations in the wells. Nonliposomal agents were added in concentration ranges of 25-500 μM (DHA) and 2.7-80 nM (MTX); liposomes were added at a lipid concentration of 1 mM (with the experimental phospholipid representing up to 15 mol % of the lipids). Control liposomes were made from 1 mM 18:0, 18:1 PC/ 18:0, 18:1 PE (9/1, mol:mol). After 6 h of incubation, 0.5 μCi/well of [³H]thymidine (2 Ci/mmol; Amersham, Arlington Heights, Ill.) was added and the incubation continued for an additional 18 h. Cells were then harvested onto glass fiber filters (PHD Cell Harvester; Cambridge Technology, Watertown, Mass.) and the radioactivity present in DNA on the filters was measured by liquid scintillation counting.

Assessing Synergism

To test whether the combined actions of DHA and MTX, added as individual agents, were additive, subadditive or superadditive (synergistic), the IC50 (dose for 50% inhibition of cell proliferation) was determined for DHA and for MTX. These two agents were then added together to cultures in doses that together theoretically produce 50% inhibition; if the observed inhibition is greater than that predicted, the agents are synergistic. The IC50 for each agent alone was determined by non-linear curve fitting with the software package SigmaPlot (SPSS, Chicago, Ill.).

### Results and Discussion

Physical Characterization of MTX-PC Derivatives

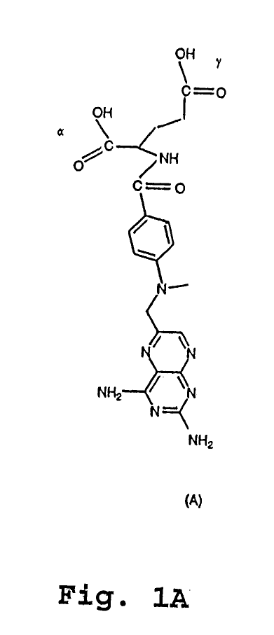

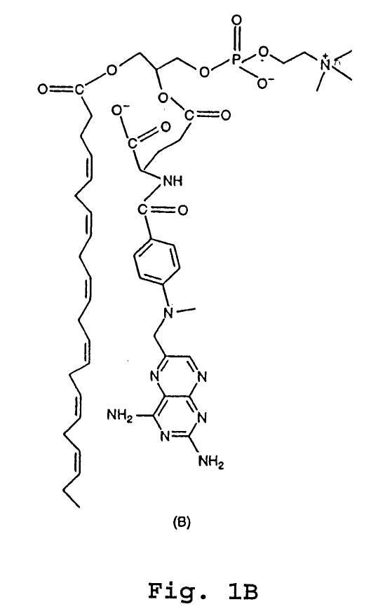

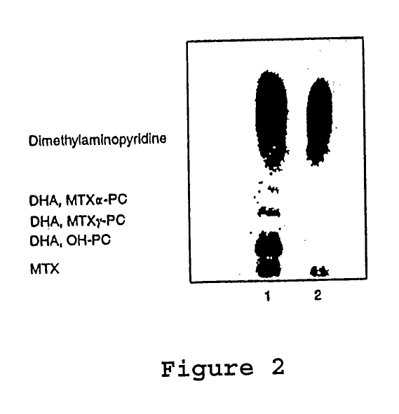

The proposed structure of the major DHA- and MTX-containing PC (22:6, MTXγ-PC) synthesized and tested in these experiments is shown in FIG. 1. We propose this structure based on the product's performance in several analyses described below. The synthesized MTX-PC derivatives were separated on thin-layer chromatography plates (FIG. 2), and the putative product bands were yellow and absorbed UV light at 254 nm (both characteristic of MTX), and gave a positive molybdate test for phosphorus. The Rf values of the bands were 0.22 and 0.35 for the 18:0, MTX-PC derivatives, and 0.30 and 0.40 for the 22:6, MTX-PC derivatives (FIG. 2). Each of these bands was isolated and characterized by the combination of techniques described below.

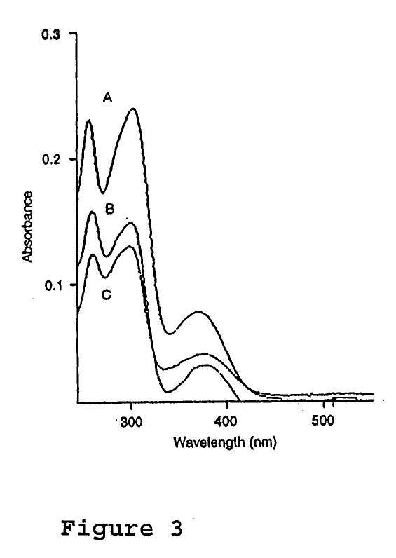

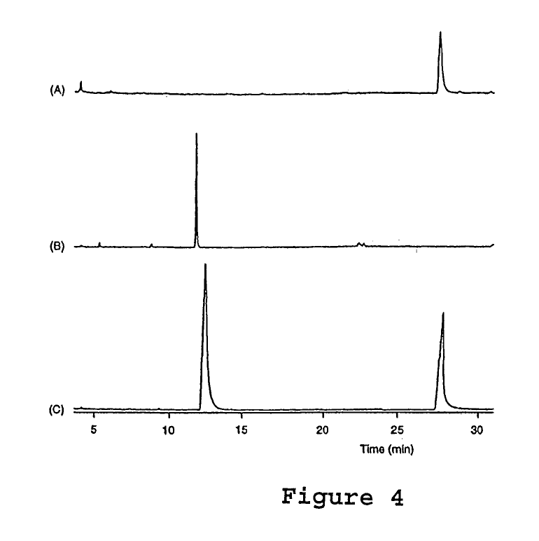

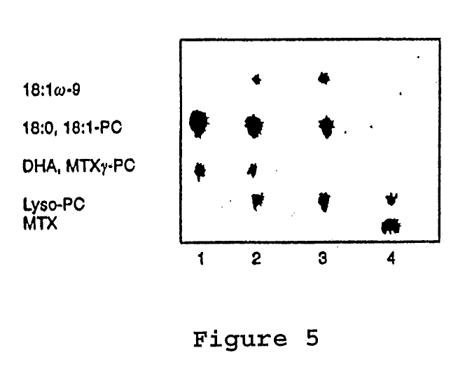

The MTX-PC structure is based on four observations. First, the absorption spectra for the products were obtained and compared to MTX (FIG. 3). The nearly identical spectra for free MTX and the 18:0, MTXγ-PC and 22:6, MTXγ-PC products had peak maxima at 260, 300 and 375 nm. Second, the reaction products were hydrolyzed, methylated and analyzed by gas chromatography. The organic-soluble 18:0, MTX-PC products gave a single chromatographic peak corresponding to stearic acid while the 22:6, MTX-PC products gave a single peak corresponding to DHA (FIG. 4). Third, we determined the amount of phosphorus and MTX for each product, and found the molar ratios of inorganic phosphorus/MTX to be between 1 and 1.2. Finally, when the reaction products were incorporated into liposomes and exposed to phospholipase A₂, they were hydrolyzed. The resulting thin-layer chromatography bands corresponded to free MTX and either 18:0, OH PC or 22:6, OH PC (FIG. 5). The sensitivity of MTX in the sn-2 position to phospholipase A₂ is particularly noteworthy, as MTX release is presumed necessary for MTX to act on cells treated with this novel phospholipid.

These results strongly indicate that the reaction of MTX with either 18:0, OH PC or 22:6, OH PC results in products containing MTX exclusively in the sn-2 position. MTX has two carboxyl groups, designated α and γ, in the glutamyl portion of the molecule that could react with the free hydroxyl of lyso-PC. The α-carboxyl group has a pKa of 3.35, while the γ-carboxyl's pKa is 4.70 (35). The slower migrating MTX-PCs, with Rf values of 0.22 (18:0, MTX-PC) and 0.30 (22:6, MTX-PC), are the γ derivatives because in these products the more polar (dissociated) group is free and would retard mobility. The products with the higher Rf values (0.35 for 18:0, MTX-PC and 0.40 for 22:6, MTX-PC) are α-carboxyl derivatives. For both 18:0, MTX-PC and 22:6, MTX-PC, the γ derivative was formed preferentially (80% of the total MTX-PCs were γ) and it was these derivatives that were tested for an inhibitory effect on T27A cell proliferation. Also, because it is believed that a free α-carboxyl group is responsible for most of the binding to the dihydrofolate receptor, (7, 36) the γ-esterified derivatives would have more biological activity.

Inhibition of Proliferation by Free DHA and MTX

Both MTX and DHA are reported to inhibit cell proliferation, and here we confirm this observation for the murine leukemia cell line T27A. Because serum albumin binds fatty acids and reduces their effectiveness in many cell function assays performed in vitro, in our experiments we used a reduced serum concentration (2% compared to the usual concentration of 10% in routine long-term cultures) . We also used a low cell density (10⁴cells/well, 5×10⁴cells/ml) to insure logarithmic cell growth. The data described below are representative of at least three experiments performed for each chemical agent.

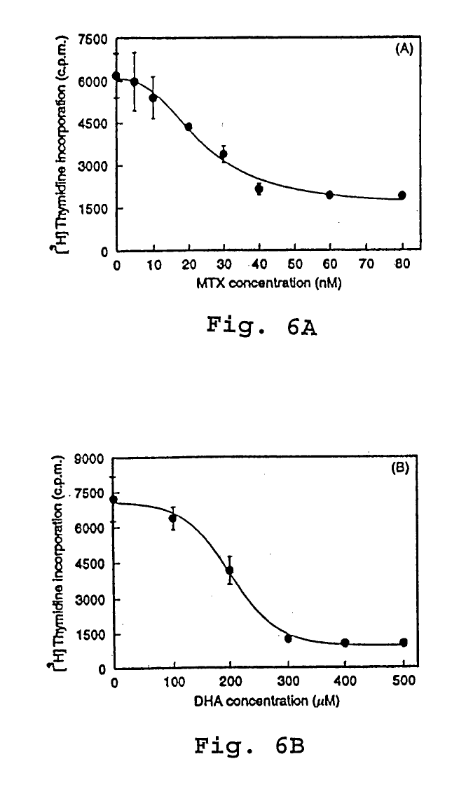

Free MTX (not conjugated to phospholipids) was added to cells in culture for 24 h and [³H]thymidine was present during the last 18 h of the incubation. The cellular incorporation of [³H]thymidine is a measure of DNA synthesis and hence an estimate of cell proliferation. FIG. 6(A) demonstrates that free MTX added to culture medium diminishes cell proliferation with an IC50 (dose for 50% inhibition of proliferation) of 30.6 nM. This is within the range of inhibitory doses, 15-240 nM, reported for MTX tested on several different cell types in a variety of assays in vitro (11, 37-39).

Under the same culture conditions, DHA (albumin-bound but not conjugated to phospholipids) inhibited [³H]thymidine Incorporation with an IC₅₀ Of 211 μM (FIG. 6B). This value is consistent with the inhibition of T27A proliferation by DHA that we have observed over the years and about 5 times higher than the IC₅₀ we obtained for DHA delivered in ethanol rather than albumin. Our observed values are similar to that reported for the human T cell leukemia Jurkat, which displayed an IC₅₀ for cell growth of 40 μM when DHA was delivered in ethanol (40).

Potential Synergism of DHA and MTX

As an antifolate, MTX Inhibits DNA synthesis during the S phase of the cell cycle. Our previous work on activated lymphocytes suggested that DHA also has some actions affecting the S phase, leading to the accumulation of cells in S phase (27). Thus, we may propose a simple model in which DHA and MTX act through different mechanisms to affect cell progression through the S phase. The question then becomes, are DHA and MTXs actions additive, superadditive (synergistic) or subadditive? From a therapeutic standpoint this is an important question; the implication is that the nutrient DHA may be used to enhance the local action of MTX, permitting the use of lower, less toxic MTX doses. Even without knowing DHA's mechanism(s) of action, we can test the hypothesis that DHA and MTX are synergistic in their inhibition of DNA synthesis (as estimated by [³H]thymidine incorporation).

T27A cells were cultured in the presence of 30 nM MTX alone, 220 μM DHA alone or combined doses of MTX and DHA. Each agent alone at the stated concentration is predicted to permit roughly halfmaximal proliferation, and this was confirmed by the observation that 30 nM MTX alone allowed 52% of maximal proliferation and 220 μM DHA, 57% (Table 1). When MTX and DHA were added concurrently to cell cultures in doses predicted to total 50% inhibition, up to 90% inhibition of proliferation was observed, implying that under certain conditions DHA and MTX are synergistic (Table 1). Synergism was most apparent at low MTX doses (2.7 and 9.5 nM) combined with relatively higher DHA doses (150 and 200 μl). At higher MTX doses, the effects of this drug and DHA appeared to be additive. These data do not provide, of course, mechanistic information but they are consistent with the idea that DHA (150-200 μM) has an action that potentiates MTX function and thus may permit very low MTX levels to be therapeutic. Because DHA is bound to albumin when delivered to cells (the molar ratio of DHA: albumin in the medium is 1.3 or lower), the actual dose of “free” fatty acid is not known. Thus, the real concentration of available DHA may be quite low, i.e. considerably closer to the MTX concentration than is immediately obvious from the total reported DHA doses (in the μM range).

TABLE 1
Interactions of MTX and DHA in the inhibition of cell proliferation

| | | | | Total | | |
|---|---|---|---|---|---|---|
| | Inhibition | | Inhibition | (Pre- | Inhibition | |
| [MTX] | (Predicted; | [DHA] | (Predicted; | dicted; | (Ob- | Inter- |
| (nm) | %) | (μM) | %) | %) | served; %) | action |
| 2.7 | 4.3 | 200 | 39.3 | 43.6 | 90.7 | synergy |
| 9.5 | 15.2 | 150 | 29.5 | 44.7 | 90.3 | synergy |
| 16.5 | 26.3 | 100 | 19.7 | 46.0 | 47.4 | additive |
| 23.3 | 37.2 | 50 | 9.8 | 47.0 | 44.6 | additive |
| 26.7 | 42.6 | 25 | 4.9 | 47.5 | 60.6 | |

Inhibition by PC Containing Both MTX and DHA

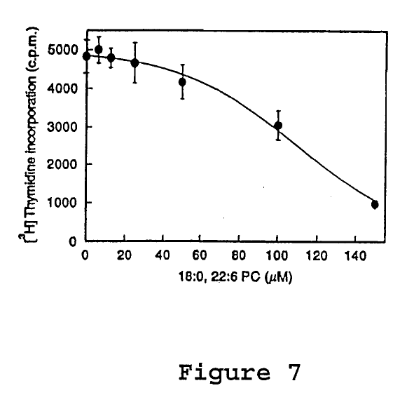

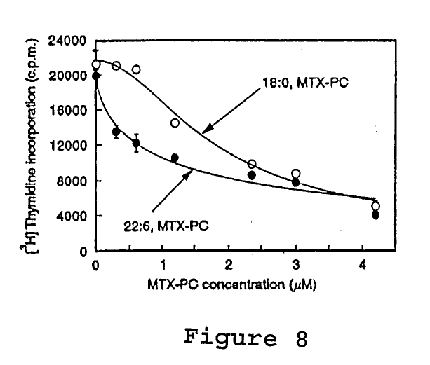

To facilitate synergism between DHA and MTX, one wishes to deliver the agents concurrently, preferably in “inactive” forms that become active once inside the target cell. The prediction is that delivery of 22:6, MTX-PC to a target cell will lead to phospholipase-mediated release of fatty acid and MTX and their synergistic or at least additive actions. To test this prediction, T27A cells were incubated with liposomes comprised primarily of 18:0, 18:1 PC (a phospholipid common in the membrane of mammalian cells) and also containing 18:0 18:1 PE (to enhance membrane fusion) plus various amounts of PC containing DHA, MTX or both. Cell proliferation relative to the control (18:0, 18:1 PC and 18:0, 18:1 PE only) was estimated by [³H]thymidine incorporation. FIG. 7 displays the inhibition of proliferation produced by inclusion of 18:0, 22:6 PC into the liposomal lipids. Inhibition was dose dependent, with an IC₅₀ of 112 μM. In contrast, as shown in FIG. 8, the MTXγ-containing PCs were much more potent, reducing cell proliferation by 70-80% at a concentration of about 4 μM compared to 150 μM for comparable inhibition by 18:0, 22:6 PC. The IC₅₀ values for stearic acid-and the DHA-containing MTXγ-PC were not greatly dissimilar, 2.11 and 1.19 μM, respectively; however, at low concentrations of the MTXγ-PCs (below 1.5 μM), the 22:6, MTXγ-PC was significantly more inhibitory than 18:0, MTXγ-PC. At doses below 25 μM, 18:0, 22:6 PC had no discernable effect on cell proliferation (FIG. 7) and therefore we would not predict DHA from 1.5 μM 22:6, MTXγ-PC to add significantly to the reduction in proliferation. One might argue that the presence of DHA in the sn-1 rather than sn-2 is position is more inhibitory. We cannot fully discount this possibility, but it is notable that in a previous study 22:6, 22:6 PC showed direct cytotoxicity comparable to 18:0, 22:6 PC (26). The implication is that the DHA, and MTX moieties of 22:6, MTXγ-PC synergize to produce inhibition greater than the sum of each active component (DHA and MTX) alone.

Synergism between fatty acids and cytotoxic or growth inhibiting drugs has been reported previously. For example, DHA potentiated toxicity of doxorubicin for glioblastoma (41) and breast cancer cells, (42) the ω-3 fatty acid eicosapentaenoic acid synergized with genistein to increase breast cancer cell apoptosis, (43) and the concurrent exposure of breast cancer cells to paclitaxel and DHA, eiciosapentaenoic acid, the ω-3 fatty acid α-linolenic acid or the ω-6 fatty acid γ-linolenic acid led to a synergistic drug interaction (44). The mechanisms for synergism are not clear and may include lipid peroxidation. Whereas cytotoxicity due to prolonged exposure of human breast cancer cells to doxorubicin and DHA was abolished by antioxidants, (42) the DHA-potentiated cytotoxicity of doxorubicin for glioblastoma cells was not caused by lipid peroxidation products (41). The involvement of lipid peroxidation in the drug potentiation mechanism depends upon cell type, levels of antioiddant enzymes (45) and the peroxidation-generating potential of the drug (42). A second, non-exclusive mechanism of action may be DHA's direct modulation of membrane structure and function (46-48). Changes in membrane structure affects membrane receptors (49) and thus may influence MTX uptake, export and intracellular transport. It would appear that when DHA is present at concentrations sufficient to induce structural changes in membranes, it can enhance the antiproliferative activity of low MTX doses.

In these experiments, the amount of the PC molecules that became incorporated into the cell, rather than adsorbed to the cell surface or remaining in suspension in the medium, is not known and therefore the true doses are not known. It is certain, however, that some liposomes do fuse with the plasma membrane, as there are changes in epitope expression on the cell surface following llposome exposure (34,50). We have additional evidence for the intimate cell association of liposomal lipids from experiments employing a fluorescence method (21) and gas chromatography (26). Although we have not yet tested this assumption directly, we expect that the fusogenicity of the two MTX-containing PCs is equivalent, as the influence of 10 mol % PE, present in all liposomal bilayers, is likely stronger than that of 0.43 mol % or less of 22:6, MTXγ-PC.

In summary, we have synthesized a novel phospholipid (22:6, MTXγ-PC) that inhibits cell proliferation as defined by the incorporation of [³H]thymidine. In future work we will determine whether this PC is rapidly cytotoxic, interferes with cell division to produce cytostasis and ultimately apoptosis or has multiple modes of action depending upon conditions including cell type. An important aspect of the current work is the suggested synergistic action between DHA, and MTX. Synergism between these two agents is indicated by experiments with the free agents (DHA and MTX); however, having these agents covalently linked to a PC and administered in liposomes insures their concurrent delivery and is amenable to targeting to specific cells. Another important implication from these studies is the possible time release of drug resulting from the action of cellular lipases. Thus, 22:6, MTXγ-PC may hold considerable promise as a chemotherapeutic agent that combines efficiency with low bystander cell toxicity.

### EXAMPLE 2

### Synthesis of Chlorambucil-PC Conjugate

The 22:6, Chlorambucil-PC conjugate was synthesized, purified and characterized similar using methods similar to those described in Example 1 for the 22:6, Methotrexate-PC congugate.

Synthesis and Purification of 18:0,Chlorambucil-PC and 22:6,Chlorambucil-PC

2 equivalents (329 μmol) of Chlorambucil (Sigma Chemical Co., St. Louis, Mo.) were dissolved in 5 ml of dry doubly distillated chloroform, after 1 hour stirring under nitrogen at room temperature, 18:0-OH-PC or 22:6-OH-PC (1 eq, 164 μmol), dicyclohexylcarbodiimide (1.5 eq, 264 μmol), and 4-dimetylaminopyridine (1 eq, μ164 mol) were added to this mixture, and stirring was continued in dark under nitrogen at room temperature for another 14 hours. For the synthesis of 22:6, Chlorambucil-PC, an anti oxidant (BHT) was added to the mixture product. To purify the novel compounds, the product of the reaction was filtered, washed with a solvent mixture of chloroform/methanol, concentrated, and resuspended in chloroform. The chloroform solution was loaded onto a caboxymethyl (CM) cellulose column, and eluted with stepwise increases of methanol in chloroform. Under these conditions, 18:0,Chlorambucil-PC or 22:6, Chlorambucil-PC was eluted at 96:4 (chloroform/methanol). The purity of the synthesized product was tested by TLC and HPLC, and the yield was estimated from phosphorus determination.

Characterization of 18:0,Chlorambucil-PC or 22:6,Chlorambucil-PC

The characterization of 18:0, Chorambucil-PC or 22:6,Chlorambucil-PC was performed by a combination of techniques as described below:

1- Crude product of the reaction was subjected to thin layer chromatography (silica gel) using a solvent mixture of (chloroform/methanol/ water/ ammonium hydroxide 28% 65/23/3/0.2) Result showed a novel formed product (Rf=0.46) as compared to the control (reaction mixture without 18:0, OH-PC). This spot absorbed UV light at 254 nm, characteristic wavelength of the chromophore Chlorambucil, and gave a positive test for phosphorus.

2- Hydrolysis of 18:0, Chlorambucil-PC or 22:6, Chlorambucil-PC using phospholipase A₂

To test the action of phospholipase A₂ on the novel compounds, small unilamellar vesicles (SUVs) were made from and 18:0,18:2-PC and 18:0, Chlorambucil-PC or 22:6, Chlorambucil-PC (80/20 mol %) in Tris pH 8.8 and the mixture was subjected to phospholipase A₂ for 20 min, The product was then extracted according to Folch. The organic phase was subjected to chromatography on silica gel plate as described above. The result showed that the hydrolysis was total showing spots for 18:0, OH-PC (Rf=0.10), Chlorambucil (Rf=0.77) and the free fatty acids. The results indicate that Chlorambucil was linked to sn-2 position of 18:0, OH-PC and acyl chain migration did not occur.

An omega-3 fatty acid, such as docosahexaenoic acid or eicosapentanoic acid may also be conjugated to this compound using the methodology provided in Example 1. The resulting conjugate can then be tested using the biological assays described in Example 1.

Conclusion

A novel phosphatidylcholine was synthesized with the polyunsaturated ω-3 fatty acid DHA in the sn-1 position and the conventional anticancer drug MTX in the sn-2 position and is described in Example 1. When included in liposomal membranes, this novel lipid inhibited leukemia cell growth in culture and displayed elements of synergism between the fatty acid and the MTX, a synergism that was also apparent when fatty acid and drug were added independently. Because this novel phosphatidylcholine may be readily included in liposome delivery vehicles, it holds great promise as a highly effective cytotoxic agent that can be targeted to cancer cells without effecting normal bystander cell viability.

Example 2 describes the synthesis and characterization of another novel lipid conjugate containing chlorambucil. It should be appreciated that any anti-cancer agent which contains the appropriate functional group for coupling to phosphatidylcholine may be included in the compositions and methods of the invention.

### REFERENCES

1. Farber S, Diamond L K, Mercer R D, Sylvester R F, Wolff J A. Temporary remissions in acute leukemia in children produced by a folic acid antagonist, 4-aminopteroyl-glutamic acid (aminopterin). New Engl J Med 1948; 238: 787-93.

2. Jolivet J, Cowan K H, Curt G A, Clendeninn H J, Chabner B A. The pharmacology and clinical use of methotrexate. New Engl J Med 1983; 309: 1094-104.

3. Wigginton S M, Chu B C F, Weisman M H, Howell S B. Methotrexate pharmacokinetics after intraarticular injection in patients with rheumatoid arthritis. Arthritis Rheum 1980; 23: 119-22.

4. Barnwarth B, Labat L, Moride Y, Schaeverbeke T. Methotrexate in rheumatoid arthritis: an update. Drugs 1994; 47: 25-50.

5. Howell S B, Krishan A, Frei E I. Cytokinetic comparison of thymidine and leucovorin rescue of marrow in humans after exposure to high dose methotrexate. Cancer Res 1979; 39: 1315-20.

6. Bertino J R. Toward improved selectivity in cancer. Cancer Res 1979; 39: 293-304.

7. Piper J R, Montgomery J A. Synthesis of α- and γ-substituted amides, peptides, and esters of methotrexate and their evaluation as inhibitors of folate metabolism. J Med Chem 1982; 25: 182-7.

8. Montgomery J A, Piper J R, Elliott R D, et al. Analogues of methotrexate. J Med Cbem 1979; 22: 862-8

9. Rosowsky A, Bader H, Kohler W, Freisheim J H, Moran R G. Methotrexate analogues. 34. Replacement of glutamate moiety in methotrexate and aminopterin by long-chain 2-amino alkanedioic acids. J Med Chem 1988; 31: 1338-44.

10. Hashimoto K, Loader J E, Kinsky S C. Synthesis and characterization of methotrexate-dimystroylethanol amine derivatives and the glycerphosphorylethanolamine analogs. Biochim Biophys Acta 1985; 816: 163-8.

11. Hashimoto K, Loader J E, Knight M S, Kinsky S C. Inhibition of cell proliferation and dihydrofolate reductase by liposomes containing methotrexatedimyristoylphoshatidylethanolamine derivatives and by the glycerophosphorylethanolamine analogs. Biochim Biophys Acta 1985; 816: 169-8.

12. Kinsky S C, Loader J E, Hashimoto K. Inhibition of cell proliferation by putative metabolites and non-degradable analogs of methotrexate-γ-dimyristoylphosphatidylethanolamine. Biochim Biophys Acta 1987; 917: 211-8.

13. Williams A S, Love W G, William B D. Synthesis of methotrexate dimyristoylphosphatidylethanolamine analogs and characterization of methotrexate release in vitro. Int J Pharmacol 1992; 85: 189-97.

14. Williams A S, Camileri J P, Goodfellow R M, Williams B D. A single intra-articular injection of liposomally conjugated methotrexate suppresses joint inflamation in rat antigen-induced arthritis. Br J Rheumatol 1996; 35: 719-24.

15. Whitting L A, Harvey C C, Century B, Worwitt M K. Dietary alterations of fatty acids of erythrocytes and mitochondria of brain and liver. J Lipid Res 1961; 2: 412-8.

16. Stillwell W. Docosahexaenoic acid and membrane lipid domains. Curr Organic Chem 2000; 4: 1169-83.

17. Galli C. Dietary omega-3 fatty acids and cancer. In: Simopoulos A P, Kifer R R, Martin R E, Barlow S M, eds. Health effects of ω3 polyunsaturated fatty acids in sea foods. New York: Karger 1991: 446-61.

18. Bums P C, Spector A& Membrane fatty acid modification in tumor cells: a potential therapeutic adjunct. Lipids 1987; 22: 178-84.

19. Lands W E M. Renewed questions about poly-unsaturated fatty acids. Nutr Rev 1986; 44: 189-95.

20. Siddiqui R A, Jenski L J, Neff K et al. Docosahexaenoic acid induces apoptosis in Jurkat cells by a protein phosphatase-mediated process. Biochim Biophys Acta 2001; 1499: 265-275.

21. Zerouga M, Stillwell W, Stone J, Powner A, Jenski L J. Phospholipid class as a determinant in docosahexaenoic acid's effect on tumor cell viability. Anticancer Res 1996; 16: 2863-8.

22. Comisky S J, Heath T D. Lealage and delivery of liposome-encapsulated methotrexate-γ-asparate in a chemically defined medium. Biochim Biophys Acta 1990; 1024: 307-17.

23. Foong W C, Green K L. Treatment of antigen-induced arthritis in rabbits with liposome-entrapped methotrexate injected intra-articularly. J Pharm Pharmacol 1993; 45: 204-9.

24. Heath T D, Lopez N G, Papahadjopoulos D. The effects of liposome size and surface charge on liposome-mediated delivery of methotrexate-γ-aspartate to cells in vitro. Biochim Biophys Acta 1985; 820: 74-84.

25. Jenski L J, Zerouga M, Stillwell W. ω-3 Fatty acid-containing liposomes in cancer therapy. Proc Soc Exp Biol Med 1995; 210: 227-33.

26. Kafawy O, Zerouga M, Stillwell W, Jenski L J. Docosahexaenoic acid in phosphatidylcholine mediates cytotoxicity more effectively than other omega-3 and omega-6 fatty acids. Cancer Lett 1998; 132: 23-9.

27. Scherer J M, Stillwell W, Jenski L J. Spleen cell survival and proliferation are differentially altered by docosahexaenoic acid. Cell Immunol 1997; 180: 153-61.

28. Zerouga M, Jenski L J, Stillwell W. Comparison of phosphatidylcholines containing one or two docosahexaenoic acyl chains on properties of phospholipid monolayers and bilayers. Biochim Biophys.)w Acta 1995; 1236: 266-72.

29. Gupta C M, Radhakrishnan R, Khorana H G. Glycerophospholipid synthesis: improved general method and new analogs containing photo-activatable groups. Proc Natl Acad Sci USA 1977; 74: 4315-9.

30. Folch J, Lees M, Sloane-Stanley G H. A simple method for the isolation and purification of total lipid from animal tissue. J Biol Chem 1957; 226: 497-509.

31. Chen P S, Toribaca T Y, Warner H. Microdetermination of phosphorus. Anal Chem 1956; 28: 1756-8.

32. Eder K, Reichlmayr-Lais A M, Kirchgessner M. Phospholipids for gas chromatographic analysis of fatty acid methyl esters. J Chromatogr 1992; 607: 55-67.

33. Spector A A, Hoak J C. An improved method for the addition of long-chain free fatty acid to protein solutions. Anal Biochem 1969; 32: 297-302.

34. Jenski L J, Bowker G M, Johnson M A, et al. Docosahexaenoic acid-induced alteration of Thy-1 and CD-8 expression on murine splenocytes. Biochim Biophys Acta 1995; 1236: 39-50.

35. Rosowsky A, Forsch R. Methotrexate analogues. 16. Importance of the side-chain amide carbonyl group as a structural determinant of biological activity. J Med Chem 1982; 25: 1454-9.

36. Rosowsky A, Freisheim J H, Bader H, et al. Methotrexate analogues. 25. Chemical and biological studies on the γ-tert-butyl esters of methotrexate and aminopterin. J Med Chem 1985; 28: 660-7.

37. Braakhuis B J, Jansen G, Noordhuis P, Kegel A, Peters G J. Importance of pharmacodynamics in the in vitro antiproliferative activity of the antifolates methotrexate and 10-ethyl-10-deazaaminopterin against human head and neck squamous cell carcinoma. Biochem Pharmacol 1993; 46: 2155-61.

38. Rosowsky A, Wright J E, Vaidya C M, et al. Synthesis and potent antifolate activity and cytotoxicity of B-ring deaza analogues of the nonpolyglutamatable dihydrofolate reductase inhibitor N alpha-(4-amino-4-deoxypteroyl)-N delta-hemiphthaloyl-_{L}-ornithine (PT523). J Med Chem 1998; 41: 5310-9.

39. Cos J, Bellmunt J, Soler C, et al. Comparative study of sequential combinations of paclitaxel and methotrexate on a human bladder cancer cell line. Cancer Invest 2000; 18: 429-35.

40. Chow S C, Sisfontes L, Bjorkhem I, Jondal M. Suppression of growth in a leukemic T cell line by n-3 and n-6 polyunsaturated fatty acids. Lipids 1989; 24: 700-4.

41. Rudra P K, Krokan H E. Cell-specific enhancement of doxorubicin toxicity in human tumour cells by docosahexaenoic acid. Anticancer Res 2001; 21: 29-38,

42. Germain E, Chajes V, Cognault S, Lhuillery C, Bougnoux P. Enhancement of doxorubicin cytotoxicity by polyunsaturated fatty acids in the human breast tumor cell line MDA-MB-231: relationship to lipid peroxidation. Internatl J Cancer 1998; 75: 578-83.

43. Nakagawa H, Yamamoto D, Kiyozuka Y, et al. Effects of genistein and synergistic action in combination with elcosapentaenoic acid on the growth of breast cancer cell lines. J Cancer Res Clin Oncol 2000; 126: 448-54.

44. Menendez J A, del Mar Barbacid M, Montero S, et al. Effects of gamma-linolenic acid and oleic acid on paclitaxel cytotoxicity in human breast cancer cells. Eur J Cancer 2001; 37: 402-13.

45. Liu Q Y, Tan B K. Effects of cis-unsaturatcd fatty acids on doxorubicin sensitivity in P388/DOX resistant and P388 parental cell lines. Life Sci 2000; 67: 1207-18.

46. Ehringer W, Belcher D, Wassall S, Stillwell W. A comparison of the effects of linolenic (18: 3Ω3) and docosahexaenoic (22: Ω63) acids on phospholipid bilayers. Chem Phys Lipids 1990; 54: 79-88.

47. Litman B J, Mitchell D C. A role for lipid polyunsaturation in modulating membrane protein function. Lipids 1996; 31: S193-197.

48. Huster D, Arnold K, Gawrisch K. Influence of docosahexaenoic acid and cholesterol on lateral lipid organization in phospholipid mixtures. Biocbemistry 1998; 37: 17299-308.

49. Jenski L J. Omega-3 fatty acids and the expression of membrane proteins: emphasis on molecules of immunologic importance. Curr Organic Chem 2000; 4: 1185-200.

50. Pascale A W, Ehringer W D, Stillwell W, Sturdevant L K, Jenski L J. Omega-3 fatty acid modification of membrane structure and function. II. Alteration by docosahexaenoic acid of tumor cell sensitivity to immune cytolysis. Nutr Cancer 1993; 19: 147-58.

While certain of the preferred embodiments of the present invention have been described and specifically exemplified above, it is not intended that the invention be limited to such embodiments. Various modifications may be made thereto without departing from the scope and spirit of the present invention, as set forth in the following claims

What is claimed is:

1. An anti-proliferative composition comprising a lipid conjugated to an ω-3 fatty acid and a chemotherapeutic agent suspended in a biologically compatible buffer.

2. The anti-proliferative composition of claim 1, wherein said ω-3 fatty acid is selected from the group consisting of docosahexanoic acid (DHA) and eicosapentaenoic acid (EPA).

3. The anti-proliferative composition of claim 1, wherein said chemotherapeutic agent is selected from the group consisting of methotrexate, chlorambucil, and melphalan.

4. The anti-proliferative composition of claim 1, wherein said lipid is selected from the group consisting of phosphatidylcholine and phosphatidylethanolamine.

5. The composition of claim 1 incorporated into a liposome.

6. The composition of claim 5, wherein said lipid is phosphatidylcholine, said ω-3 fatty acid is DHA and said chemotherapeutic agent is methotrexate.

7. The composition of claim 6, wherein said DHA is conjugated to phosphatidylcholine at the sn-1 position and said methotrexate is conjugated to phosphatidylcholine at the sn-2 position.

8. The composition of claim 6, wherein said DHA is conjugated to phosphatidylcholine at the sn-2 position and said methotrexate is conjugated to phosphatidylcholine at the sn-1 position.

9. The composition of claim 5, wherein said lipid is phosphatidylcholine, said ω-3 fatty acid is DHA and said chemotherapeutic agent is chlorambucil.

10. The composition of claim 9, wherein said DHA is conjugated to phosphatidylcholine at the sn-1 position and said chlorambucil is conjugated to phosphatidylcholine at the sn-2 position.

11. The composition of claim 9, wherein said DHA is conjugated to phosphatidylcholine at the sn-2 position and said chlorambucil is conjugated to phosphatidylcholine at the sn-1 position.

12. The composition of claim 5, wherein said lipid is phosphatidylcholine, said ω-3 fatty acid is DHA and said chemotherapeutic agent is melphalan.

13. The composition of claim 12, wherein said DHA is conjugated to phosphatidylcholine at the sn-1 position and said melphalan is conjugated to phosphatidylcholine at the sn-2 position.

14. The composition of claim 12, wherein said DHA is conjugated to phosphatidylcholine at the sn-2 position and said melphalan is conjugated to phosphatidylcholine at the sn-1 position.

15. The composition of claim 5, wherein said lipid is phosphatidylcholine, said ω-3 fatty acid is EPA and said chemotherapeutic agent is methotrexate.

16. The composition of claim 15, wherein said EPA is conjugated to phosphatidylcholine at the sn-1 position and said methotrexate is conjugated to phosphatidylcholine at the sn-2 position.

17. The composition of claim 15, wherein said EPA is conjugated to phosphatidylcholine at the sn-2 position and said methotrexate is conjugated to phosphatidylcholine at the sn-1 position.

18. The composition of claim 5, wherein said lipid is phosphatidylcholine, said ω-3 fatty acid is EPA and said chemotherapeutic agent is chlorambucil.

19. The composition of claim 18, wherein said EPA is conjugated to phosphatidylcholine at the sn-1 position and said chlorambucil is conjugated to phosphatidylcholine at the sn-2 position.

20. The composition of claim 18, wherein said EPA is conjugated to phosphatidylcholine at the sn-2 position and said chlorambucil is conjugated to phosphatidylcholine at the sn-1 position.

21. The composition of claim 5, wherein said lipid is phosphatidylcholine, said ω-3 fatty acid is EPA and said chemotherapeutic agent is melphalan.

22. The composition of claim 21, wherein EPA is conjugated to phosphatidylcholine at the sn-1 position and said melphalan is conjugated to phosphatidylcholine at the sn-2 position.

23. The composition of claim 21, wherein said EPA is conjugated to phosphatidylcholine at the sn-2 position and said melphalan is conjugated to phosphatidylcholine at the sn-1 position.

24. A method for inhibiting tumor cell growth, comprising administration of an effective amount of the composition of claim 1.
